(19) United States
(12) Patent Application Publication
(10) Pub. No.: US 2005/0215543 A1
(43) Pub. Date: Sep. 29, 2005

(54) Methods of treating HIV infection

(76) Inventors: Pin-Fang Lin, Branford, CT (US); Beata Nowicka-Sans, Newington, CT (US); Gregory Yamanaka, Middletown, CT (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 11/057,667

(22) Filed: Feb. 14, 2005

Related U.S. Application Data
(60) Provisional application No. 60/555,847, filed on Mar. 24, 2004.

(51) Int. Cl. A61K 31/551; A61K 31/496; A61K 31/522

(52) U.S. Cl. 514/220; 514/253.04; 514/263.31

(57) ABSTRACT

The invention encompasses pharmaceutical compositions and methods for treating AIDS and HIV infection employing Compound 1.

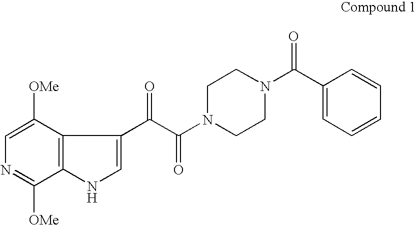

### CROSS-REFERENCE TO RELATED APPLICATIONS

This application claims the benefit of U.S. provisional application U.S. Ser. No. 60/555,847, filed Mar. 24, 2004.

### BACKGROUND OF THE INVENTION

HIV-1 (human immunodeficiency virus-1) infection remains a major medical problem, with an estimated 42 million people infected worldwide at the end of 2002. The number of cases of HIV and AIDS (acquired immunodeficiency syndrome) has risen rapidly. In 2002, approximately 5 million new infections were reported and 3.1 million people died from AIDS. Currently available drugs for the treatment of HIV include ten nucleoside reverse transcriptase (RT) inhibitors or approved single pill combinations: zidovudine or AZT (or Retrovir®), didanosine or DDI (or Videx®), stavudine or D4T (or Zerit®), lamivudine or 3TC (or Epivir®), zalcitabine or DDC (or Hivid®), abacavir succinate (or Ziagen®), tenofovir disoproxil fumarate salt (or Viread®), emtricitabine (or Emtriva®), Combivir® (contains 3TC and AZT), Trizivir® (contains abacavir, 3TC and AZT); three non-nucleoside reverse transcriptase inhibitors: nevirapine (or Viramune®), delavirdine (or Rescriptor®) and efavirenz (or Sustiva®), eight peptidomimetic protease inhibitors or approved formulations: saquinavir (or Invirase® or Fortovase®), indinavir (or Crixivan®), ritonavir (or Norvir®), nelfinavir (or Viracept®), amprenavir (or Agenerase®), atazanavir (Reyataz®), fosamprenavir (or Lexiva), Kaletra® (contains lopinavir and ritonavir), and one fusion inhibitor enfuvirtide (or T-20 or Fuzeon®).

Each of these drugs can only transiently restrain viral replication if used alone. However, when used in combination, these drugs have a profound effect on viremia and disease progression. In fact, significant reductions in death rates among AIDS patients have been recently documented as a consequence of the widespread application of combination therapy. Despite these impressive results, 30 to 50% of patients ultimately fail combination drug therapies. Insufficient drug potency, non-compliance, restricted tissue penetration and drug-specific limitations within certain cell types (e.g. most nucleoside analogs cannot be phosphorylated in resting cells) may account for the incomplete suppression of sensitive viruses. Furthermore, the high replication rate and rapid turnover of HIV-1 combined with the frequent incorporation of mutations, leads to the appearance of drug-resistant variants and treatment failures when sub-optimal drug concentrations are present (Larder and Kemp; Gulick; Kuritzkes; Morris-Jones et al; Schinazi et al; Vacca and Condra; Flexner; Berkhout and Ren et al; (Ref. 6-14)). Thus, there is continuing need for new compounds and methods of treatment for HIV infection.

1-Benzoyl-4-[2-(4,7-dimethoxy-11H-pyrrolo[2,3-c]pyridin-3-yl)-1,2-dioxoethyl]-piperazine (Compound 1, N-(benzoyl)-N′-[(4,7-dimethoxy-6-azaindol-3-yl)-oxoacetyl]piperazine) is an HIV-1 attachment inhibitor demonstrating potent antiviral activity against a variety of laboratory and clinical strains of HIV-1 (see U.S. Pat. No. 6,476,034; U.S. Pat. No. 6,632,819; Hanna et al., Abstract 141 presented at the 11th Conference on Retroviruses and Opportunistic Infections, San Francisco, Calif., Feb. 8-11, 2004; Lin et al., Poster 534 presented at the 11th Conference on Retroviruses and Opportunistic Infections, San Francisco, Calif., Feb. 8-11, 2004; Hanna et al., Poster 535 presented at the 11th Conference on Retroviruses and Opportunistic Infections, San Francisco, Calif., Feb. 8-11, 2004). U.S. Pat. No. 6,476,034 and U.S. Pat. No. 6,632,819 are herein incorporated by reference in their entirety.

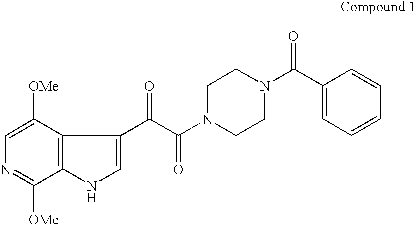

Compound 1 acts by selectively preventing attachment of the exterior viral envelope protein gp120 to its cellular receptor CD4. Binding of gp120 to CD4 is the first step in viral entry and is distinct from the subsequent interaction with a chemokine receptor (CCR5 or CXCR4) or virus-cell fusion event. By inhibiting this interaction, Compound 1 blocks viral entrance into cells.

### DESCRIPTION OF THE INVENTION

The invention encompasses pharmaceutical compositions and methods for treating HIV infection and AIDS.

One aspect of the invention is a method for treating HIV infection in a human patient comprising the administration of a therapeutically effective amount of 1-benzoyl-4-[2-(4,7-dimethoxy-1H-pyrrolo[2,3-c]pyridin-3-yl)-1,2-dioxoethyl]-piperazine (Compound 1), or a pharmaceutically acceptable salt or solvate thereof, with a therapeutically effective amount of at least one other agent used for treatment of AIDS or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

Another aspect of the invention is a method wherein the agent is a nucleoside HIV reverse transcriptase inhibitor.

Another aspect of the invention is a method wherein the nucleoside HIV reverse transcriptase inhibitor is selected from the group consisting of abacavir, didanosine, emtricitabine, lamivudine, stavudine, tenofovir, zalcitabine, and zidovudine, or a pharmaceutically acceptable salt or solvate thereof.

Another aspect of the invention is a method wherein the agent is a non-nucleoside HIV reverse transcriptase inhibitor.

Another aspect of the invention is a method wherein the non-nucleoside HIV reverse transcriptase inhibitor is selected from the group consisting of delavirdine, efavirenz, and nevirapine, or a pharmaceutically acceptable salt or solvate thereof.

Another aspect of the invention is a method wherein the agent is an HIV protease inhibitor.

Another aspect of the invention is a method wherein the HIV protease inhibitor is selected from the group consisting of amprenavir, atazanavir, indinavir, lopinavir, nelfinavir, ritonavir, saquinavir and fosamprenavir, or a pharmaceutically acceptable salt or solvate thereof.

Another aspect of the invention is a method wherein the agent is an HIV fusion inhibitor.

Another aspect of the invention is a method wherein the HIV fusion inhibitor is enfuvirtide or T-1249, or a pharmaceutically acceptable salt or solvate thereof.

Another aspect of the invention is a method wherein the agent is an HIV attachment inhibitor.

Another aspect of the invention is a method wherein the agent is a CCR5 inhibitor.

Another aspect of the invention is a method wherein the CCR5 inhibitor is selected from the group consisting of Sch-C, Sch-D, TAK-220, PRO-140, and UK-427,857, or a pharmaceutically acceptable salt or solvate thereof.

Another aspect of the invention is a method wherein the agent is a CXCR4 inhibitor.

Another aspect of the invention is a method wherein the CXCR4 inhibitor is AMD-3100, or a pharmaceutically acceptable salt or solvate thereof.

Another aspect of the invention is a method wherein the agent is an HIV budding or maturation inhibitor.

Another aspect of the invention is a method wherein the budding or maturation inhibitor is PA-457, or a pharmaceutically acceptable salt, or solvate thereof.

Another aspect of the invention is a method wherein the agent is an HIV integrase inhibitor.

Another aspect of the invention is a method wherein the HIV integrase inhibitor is 3-[(4-fluorobenzyl)methoxycarbamoyl]-2-hydroxyacrylic acid (Compound 2) or 2-(2,2)-dimethyl-5-oxo-[1,3]-dioxolan-4-ylidene)-N-(4-fluorobenzyl)-N-methoxyacetamide (Compound 3), or a pharmaceutically acceptable salt or solvate thereof.

Another aspect of the invention is a pharmaceutical composition comprising a therapeutically effective amount of 1-benzoyl-4-[2-(4,7-dimethoxy-1H-pyrrolo[2,3-c]pyridin-3-yl)-1,2-dioxoethyl]-piperazine, or a pharmaceutically acceptable salt or solvate thereof, with at least one other agent used for treatment of AIDS or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier.

Another aspect of the invention is the composition wherein the agent is a nucleoside HIV reverse transcriptase inhibitor.

Another aspect of the invention is the composition wherein the nucleoside HIV transcriptase inhibitor is selected from the group consisting of abacavir, didanosine, emtricitabine, lamivudine, stavudine, tenofovir, zalcitabine, and zidovudine, or a pharmaceutically acceptable salt or solvate thereof.

Another aspect of the invention is the composition wherein the agent is a non-nucleoside HIV reverse transcriptase inhibitor.

Another aspect of the invention is the composition wherein the non-nucleoside HIV reverse transcriptase inhibitor is selected from the group consisting of delavirdine, efavirenz, and nevirapine, or a pharmaceutically acceptable salt or solvate thereof.

Another aspect of the invention is the composition wherein the agent is an HIV protease inhibitor.

Another aspect of the invention is the composition wherein the HIV protease inhibitor is selected from the group consisting of amprenavir, atazanavir, indinavir, lopinavir, nelfinavir, ritonavir, saquinavir and fosamprenavir, or a pharmaceutically acceptable salt or solvate thereof.

Another aspect of the invention is the composition wherein the agent is an HIV fusion inhibitor.

Another aspect of the invention is the composition method wherein the HIV fusion inhibitor is enfuvirtide or T-1249, or a pharmaceutically acceptable salt or solvate thereof.

Another aspect of the invention is the composition wherein the agent is an HIV attachment inhibitor.

Another aspect of the invention is the composition wherein the agent is a CCR5 inhibitor.

Another aspect of the invention is the composition wherein the CCR5 inhibitor is selected from the group consisting of Sch-C, Sch-D, TAK-220, PRO-140, and UK-427,857, or a pharmaceutically acceptable salt or solvate thereof.

Another aspect of the invention is a method wherein the agent is a CXCR4 inhibitor.

Another aspect of the invention is a method wherein the CXCR4 inhibitor is AMD-3100 or a pharmaceutically acceptable salt or solvate thereof.

Another aspect of the invention is the composition wherein the agent is an HIV budding or maturation inhibitor.

Another aspect of the invention is the composition wherein the budding or maturation inhibitor is PA-457, or a pharmaceutically acceptable salt or solvate thereof.

Another aspect of the invention is the composition wherein the agent is an HIV integrase inhibitor.

Another aspect of the invention is the composition wherein the HIV integrase inhibitor is 3-[(4-fluorobenzyl)methoxycarbamoyl]-2-hydroxyacrylic acid (Compound 2) or 2-(2,2)-dimethyl-5-oxo-[1,3]-dioxolan-4-ylidene)-N-(4-fluorobenzyl)-N-methoxyacetamide (Compound 3), or a pharmaceutically acceptable salt or solvate thereof.

“Combination,” “coadministration,” “concurrent,” and similar terms referring to the administration of Compound 1 with at least one anti-HIV agent mean that the components are part of a combination antiretroviral therapy or highly active antiretroviral therapy (HAART) as understood by practitioners in the field of AIDS and HIV infection.

“Therapeutically effective” means the amount of agent required to provide a meaningful patient benefit as understood by practitioners in the field of AIDS and HIV infection. In general, the goals of treatment are suppression of viral load, restoration and preservation of immunologic function, improved quality of life, and reduction of HIV-related morbidity and mortality.

“Patient” means a person infected with the HIV virus and suitable for therapy as understood by practitioners in the field of AIDS and HIV infection.

“Treatment,” “therapy,” “regimen,” “HIV infection,” “ARC,” “AIDS” and related terms are used as understood by practitioners in the field of AIDS and HIV infection.

The invention includes all pharmaceutically acceptable salt forms of Compound 1. Pharmaceutically acceptable salts are those in which the counter ions do not contribute significantly to the physiological activity or toxicity of the compounds and as such function as pharmacological equivalents. In many instances, salts have physical properties that make them desirable for formulation, such as solubility or crystallinity. The salts can be made according to common organic techniques employing commercially available reagents. Suitable anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate.

The invention also includes all solvated forms of Compound 1, particularly hydrates. Solvates do not contribute significantly to the physiological activity or toxicity of the compounds and as such function as pharmacological equivalents. Solvates may form in stoichiometric amounts or may form from adventitious solvent or a combination of both. One type of solvate is hydrate. Some hydrated forms include monohydrate, hemihydrate, and dihydrate.

### Biological Methods

Compound 1 demonstrated synergistic or additive-synergistic HIV antiviral activity when used in conjunction with a variety of other antiviral agents, as described below.

Virus and cell lines. The T-cell lines, MT-2 and PM-1 were obtained through the AIDS Research and Reference Reagent Program, NIAID, and were contributed by Dr. D. Richman and Dr. R. Gallo, respectively. Both cell lines were cultured in RPMI 1640 medium supplemented with 10% fetal bovine serum, 2 mM L-glutamine and sub-cultured twice a week. The LAI strain of HIV-1 was obtained from the Fred Hutchinson Cancer Research Center, and the Bal strain was from NIH. Both virus stocks were amplified and titered in MT-2 cells (LAI) and PM-1 cells (Bal) using a virus infectivity assay.

Chemicals. Compound 1, atazanavir, didanosine, stavudine, efavirenz, enfuvirtide (T-20), T-1249, AMD-3100, Sch-C, Sch-D and UK-427,857 were synthesized using published or known reactions. Amprenavir, indinavir, nelfinavir, nevirapine, lopinavir, lamivudine, ritonavir, tenofovir, saquinavir, delavirdine and abacavir were extracted from commercial formulations of the prescribed drugs and purified using published or common techniques. Tenofovir was tested as tenovir disopoxil fumerate. Zalcitabine was obtained from the National Institutes of Health. Zidovudine was purchased from Sigma and emtricitabine from Moravek Biochemicals. 3-[(4-Fluorobenzyl)methoxycarbamoyl]-2-hydroxyacrylic acid (Compound 2) and 2-(2,2)-dimethyl-5-oxo-[1,3]-dioxolan-4-ylidene)-N-(4-fluorobenzyl)-N-methoxyacetamide (Compound 3) are described in U.S. Pat. No. 6,777,440. Purities of the anti-HIV agents were greater than 95% except for AMD-3100 (>90%), Sch-D (80%), and UK-427,857 (>90%).

Drug Susceptibility and Cytotoxicity Assays. For drug susceptibility assays, MT-2 cells were infected with HIV-1 LAI (or PM-1 cells with HIV-1 Bal) at an MOI of 0.005, and seeded into 96-well microtiter plates (0.1×10⁶ cells/ml) containing serial dilutions of test compounds. The drug combinations were set up using ratios of the two drugs of 1:1, 1:2.5 and 2.5:1 times the EC₅₀ value determined for each drug in prior multiple experiments. Each drug ratio consisted of an array of 3-fold serial dilutions, and was performed in quadruplicate. The plates were incubated at 37° C./5% CO₂. The MT-2 cells infected with HIV-1 LAI were incubated for 5 days. On day-five post-infection, 20 μl from each well was harvested and quantitated by a reverse transcriptase (RT) assay, or in samples involving non-nucleoside RT inhibitors, an MTS assay. The PM-1 cells infected with HIV-1 Bal and used for studying the combinations with CCR5 inhibitors were incubated for six days. On day-six post-infection, 20 μl from each well was harvested, 20- and 50-fold diluted and quantitated by p24 assay. Cytotoxicity assays were performed using uninfected cells, exposed to the same drug combinations, and incubated for six days. Cell viability was determined by an XTT assay. The CC₅₀ values were calculated by using the exponential form of the median effect equation as mentioned below for calculation of EC₅₀.

Analysis of Drug Combination Effects. For determination of CI values, drugs were diluted in a fixed ratio and multiple ratios were analyzed. The drug serial dilutions spanned a range of concentrations near the EC₅₀ value of each compound, so that equivalent antiviral activities could be compared. Concentration-response curves were estimated for each individual drug and every combination using the median-effect equation. The equation was fit using a nonlinear regression routine (Proc Nlin) in PC SAS version 8.01 (SAS Institute Inc., SAS Version 8.01, Cary, N.C.: SAS Institute Inc., 1990).

EC₅₀ values for each drug were determined from the single drug experiments, using the median effect equation, Fa=1/[1+(ED₅₀/drug concentration)^{m}]. In this equation, Fa stands for “fraction affected,” and represents the fraction of the viral load that has been inactivated. For example, Fa of 0.75 indicates that viral replication had been inhibited by 75%, relative to the no-drug controls. ED₅₀ is drug concentration that is expected to reduce the amount of virus by 50%, and m is a parameter that reflects the slope of the concentration-response curve.

To assess antiviral effects of different drug combination treatments, combination indices (CIs) were calculated according to Chou and Rideout. The combination index was computed as
CI=[D]_{1/[Dm]1+}[D]_{2/[Dm]2}

In this equation [Dm]1 and [Dm]2 are the concentrations of drugs that would individually produce a specific level of effect, while [D]1 and [D]2 are the concentrations of drugs in combination that would produce the same level of effect.

Theoretically, additivity is implied if the CI is equal to one, synergy if the CI is less than one, and antagonism if the CI is greater than one. However, extensive experience with combination studies indicates that there are inherent laboratory variables that must be taken into account in interpreting the CIs. At best, we can construct a range that contains the likely values for the CI, given the noise in the data. In this report, these ranges are reported in parentheses next to each point estimate of the CI. For example, when we report a CI of “0.53 (0.46, 0.60)” this means that our best estimate of the CI is 0.53, but due to noise in the data, values from 0.46 to 0.60 are also reasonable values for the CI. This range, 0.46 to 0.60 falls entirely below the value of 1.0, and hence all likely values for the CI are less than 1.0. Therefore, we can infer synergistic behavior for this case. If the range fell entirely above 1.0, we would infer antagonistic behavior. If the range were to include 1.0, we would infer additivity.

In carrying out the combination experiments below, the EC₅₀ for Compound 1 and each comparator compound was determined during the course of each study, and used in the subsequent data analysis. The determined values are consistent with our previously published data and are shown in Table 1.

TABLE 1
Anti-HIV Activity of the Compounds Used
in Two-Drug Combination Studies

| | | Highest Conc. Used |
|---|---|---|
| Compound | EC₅₀ (μM) | (μM) |
| Compound 1 | 0.001-0.004 | 1.0 |
| Abacavir | 0.40 | 150 |
| Tenofovir | 0.016 | 6.0 |
| Zalcitabine | 0.11 | 30 |
| Compound 1 | 0.001-0.004 | 1.0 |
| Didanosine | 1.8 | 300 |
| Stavudine | 0.25 | 100 |
| Zidovudine | 0.003 | 0.9 |
| Lamivudine | 0.14 | 6.0 |
| Emtricitabine | 0.034 | 30 |
| Efavirenz | 0.00006 | 1.0 |
| Nevirapine | 0.063 | 24 |
| Delavirdine | 0.0016 | 30 |
| Indinavir | 0.0007 | 3.0 |
| Atazanavir | 0.001 | 0.9 |
| Lopinavir | 0.001 | 6.0 |
| Nelfinavir | 0.022 | 3.0 |
| Amprenavir | 0.021 | 6.0 |
| Saquinavir | 0.009 | 3.0 |
| Ritonavir | | |
| Enfuvirtide | 0.003 | 5.3 |
| T-1249 | 0.0004 | 0.6 |
| AMD-3100 | 0.008 | 6.0 |
| Sch-C | 0.003 | 0.9 |
| Sch-D | 0.0005 | 0.15 |
| UK-427,857 | | |
| Compound 2 | 0.039 | 30 |

Two-Drug Combinations of Compound 1 with Nucleoside Reverse Transcriptase Inhibitors. Eight nucleoside RT inhibitors (didanosine, stavudine, zidovudine, lamivudine, abacavir, zalcitabine, emtricitibine and the nucleoside phosphonate tenofovir) were combined with Compound 1 at a range of concentrations near the EC₅₀ value of each compound, so that equivalent antiviral activities could be compared. All estimates were computed using SAS Proc NLIN, and a two-parameter logistic. Data is presented in Table 2 as the combination indices and the asymptotic confidence intervals for RT inhibitors at different molar ratios (see Materials and Methods).

Four nucleoside RT inhibitors, abacavir, tenofovir, zalcitabine and emtricitibine show synergistic antiviral effects in combination with Compound 1. Tenofovir, zalcitabine and emtricitabine show consistent synergy at all effective levels and concentration ratios. Abacavir exhibits strong synergy at the 50% and 75% effective levels, but approaches additivity at the 90% HIV-1 inhibition. Therefore, the overall result is estimated as moderate synergy. Didanosine and stavudine exhibit synergy only at the 75% and 90% effective levels. These latter compounds are therefore classified as additive-synergistic. Combining zidovudine with Compound 1 yields additive to synergistic interactions, with a bias toward synergy at the 1:04 molar ratio. Lamivudine shows strong synergy at the 75% and 90% effective levels and additivity at the 50% effective levels, for all three molar ratios tested. Taking all the CI values and the analyses into account, the overall effect of combining nucleoside RT inhibitors with Compound 1 is mostly additive to synergistic. No significant antagonism of anti-HIV activity is observed. No enhanced cytotoxicity was encountered at the highest concentrations tested with any of the drug combinations, as measured by XTT reduction assay.

TABLE 2
Two-Drug Combinations using Compound 1 and Nucleoside
Reverse Transcriptase Inhibitors.

| | Combination Indices at % HIV Inhibition^{b} | | | |
|---|---|---|---|---|
| Molar Ratio | (Confidence Interval) | | | Overall |
| (EC₅₀ Ratio)^{a} | 50% | 75% | 90% | Result |

Tenofovir

| | | | | | |
|---|---|---|---|---|---|
| 1:6.7 | (1:1) | 0.51 (0.44, 0.58) | 0.43 (0.35, 0.52) | 0.42 (0.30, 0.55) | Synergistic |
| 1:16.7 | (1:2.5) | 0.93 (0.79, 1.07) | 0.53 (0.43, 0.63) | 0.34 (0.24, 0.44) | |
| 1:2.7 | (2.5:1) | 0.80 (0.75, 0.86) | 0.52 (0.49, 0.56) | 0.38 (0.35, 0.41) | |

Zalcitabine

| | | | | | |
|---|---|---|---|---|---|
| 1:33.3 | (1:1) | 0.55 (0.41, 0.70) | 0.57 (0.36, 0.77) | 0.59 (0.25, 0.92) | Synergistic |
| 1:83.3 | (1:2.5) | 0.87 (0.65, 1.08) | 0.66 (0.44, 0.89) | 0.51 (0.24, 0.78) | |
| 1:13.3 | (2.5:1) | 0.68 (0.48, 0.89) | 0.64 (0.37, 0.90) | 0.59 (0.20, 0.98) | |

Emtricitabine

| | | | | | |
|---|---|---|---|---|---|
| 1:200 | (1:1) | 0.57 (0.49, 0.64) | 0.33 (0.27, 0.39) | 0.19 (0.14, 0.24) | Synergistic |
| 1:500 | (1:2.5) | 0.38 (0.31, 0.45) | 0.48 (0.36, 0.60) | 0.60 (0.36, 0.84) | |
| 1:80 | (2.5:1) | 0.17 (0.15, 0.19) | 0.21 (0.17, 0.25) | 0.26 (0.18, 0.34) | |

Abacavir

| | | | | | |
|---|---|---|---|---|---|
| 1:167 | (1:1) | 0.29 (0.16, 0.42) | 0.51 (0.20, 0.82) | 0.91 (0.03, 1.78) | Moderate |
| 1:417 | (1:2.5) | 0.53 (0.36, 0.70) | 0.61 (0.33, 0.88) | 0.71 (0.21, 1.22) | Synergistic |
| 1:67 | (2.5:1) | 0.36 (0.27, 0.45) | 0.52 (0.34, 0.71) | 0.76 (0.34, 1.19) | |

Didanosine

| | | | | | |
|---|---|---|---|---|---|
| 1:333 | (1:1) | 1.29 (1.18, 1.40) | 0.69 (0.60, 0.78) | 0.37 (0.29, 0.44) | Additive- |
| 1:833 | (1:2.5) | 0.53 (0.46, 0.60) | 0.32 (0.26, 0.38) | 0.19 (0.13, 0.25) | Synergistic |
| 1:133 | (2.5:1) | 0.95 (0.74, 1.17) | 0.69 (0.48, 0.90) | 0.50 (0.26, 0.74) | |

Stavudine

| | | | | | |
|---|---|---|---|---|---|
| 1:67 | (1:1) | 1.47 (1.16, 1.79) | 0.60 (0.42, 0.78) | 0.25 (0.13, 0.38) | Additive- |
| 1:167 | (1:2.5) | 0.50 (0.37, 0.64) | 0.29 (0.18, 0.40) | 0.18 (0.07, 0.28) | Synergistic |
| 1:27 | (2.5:1) | 0.69 (0.47, 0.91) | 0.55 (0.30, 0.80) | 0.45 (0.12, 0.78) | |

Zidovudine

| | | | | | |
|---|---|---|---|---|---|
| 1:1 | (1:1) | 1.06 (0.85, 1.27) | 0.99 (0.72, 1.26) | 0.93 (0.53, 1.34) | Additive- |
| 1:2.5 | (1:2.5) | 1.21 (1.01, 1.40) | 0.95 (0.74, 1.16) | 0.76 (0.50, 1.02) | Synergistic |
| 1:0.4 | (2.5:1) | 0.81 (0.69, 0.92) | 0.70 (0.56, 0.83) | 0.60 (0.42, 0.79) | |

Lamivudine

| | | | | | |
|---|---|---|---|---|---|
| 1:6.7 | (1:1) | 1.02 (0.80, 1.23) | 0.59 (0.42, 0.76) | 0.37 (0.21, 0.53) | Additive- |
| 1:16.7 | (1:2.5) | 0.89 (0.76, 1.02) | 0.41 (0.34, 0.47) | 0.20 (0.15, 0.24) | Synergistic |
| 1:2.7 | (2.5:1) | 1.07 (0.95, 1.20) | 0.71 (0.58, 0.83) | 0.50 (0.36, 0.65) | |

^{a}Ratio of Compound 1 to comparator compound.
^{b}A lower bound of the asymptotic confidence interval greater than 1 indicates antagonisms, an upper bound of less than 1 indicates synergism, and a value of 1 being contained in the interval indicates additivity. The 95% confidence intervals are shown in parenthesis, and represent a measure of variability in the data.

Two-Drug Combinations of Compound 1 with Non-Nucleoside Reverse Transcriptase Inhibitors. Three non-nucleoside RT inhibitors were combined with Compound 1 at a range of concentrations near the EC₅₀ value of each compound, as described above for nucleoside RT inhibitors. Data is presented in Table 3 as the combination indices and the asymptotic confidence intervals at different molar ratios. All three compounds, efavirenz, nevirapine, and delavirdine show strong synergistic effects in combination with Compound 1. Synergy is seen at all effective concentrations and at all molar ratios. No enhanced cytotoxicity was observed at the highest concentrations tested with any of the drug combinations.

TABLE 3
Two-Drug Combinations using Compound 1 and Non-Nucleoside
Reverse Transcriptase Inhibitors

| | Combination Indices at % HIV Inhibition^{b} | | | |
|---|---|---|---|---|
| Molar Ratio | (Confidence Interval) | | | Overall |
| (EC₅₀ Ratio)^{a} | 50% | 75% | 90% | Result |

Efavirenz

| | | | | | |
|---|---|---|---|---|---|
| 1:0.33 | (1:1) | 0.71 (0.50, 0.91) | 0.64 (0.38, 0.90) | 0.64 (0.22, 1.06) | Synergistic |
| 1:0.83 | (1:2.5) | 0.23 (0.17, 0.29) | 0.23 (0.17, 0.30) | 0.25 (0.12, 0.38) | |
| 1:0.13 | (2.5:1) | 0.58 (0.45, 0.70) | 0.47 (0.32, 0.61) | 0.46 (0.23, 0.69) | |

Nevirapine

| | | | | | |
|---|---|---|---|---|---|
| 1:27 | (1:1) | 0.87 (0.79, 0.94) | 0.80 (0.69, 0.90) | 0.74 (0.58, 0.89) | Synergistic |
| 1:67 | (1:2.5) | 0.85 (0.74, 0.96) | 0.68 (0.58, 0.79) | 0.55 (0.43, 0.66) | |
| 1:10.6 | (2.5:1) | 0.71 (0.62, 0.81) | 0.63 (0.52, 0.73) | 0.56 (0.42, 0.70) | |

Delavirdine

| | | | | | |
|---|---|---|---|---|---|
| 1:33 | (1:1) | 0.11 (0.00, 0.26) | 0.09 (0.02, 0.15) | 0.09 (0.00, 0.21) | Synergistic |
| 1:83 | (1:2.5) | 0.32 (0.20, 0.43) | 0.09 (0.08, 0.10) | 0.03 (0.02, 0.03) | |
| 1:13 | (2.5:1) | 0.28 (0.22, 0.35) | 0.17 (0.14, 0.20) | 0.16 (0.11, 0.35) | |

^{a}Ratio of Compound 1 to comparator compound.
^{b}A lower bound of the asymptotic confidence interval greater than 1 indicates antagonisms, an upper bound of less than 1 indicates synergism, and a value of 1 being contained in the interval indicates additivity. The 95% confidence intervals are shown in parenthesis, and represent a measure of variability in the data.

Two-Drug Combinations Involving Compound 1 and HIV Protease Inhibitors. Evaluation of Compound 1 for drug combination therapy with protease inhibitors was carried out using indinavir, amprenavir, nelfinavir, lopinavir, saquinavir and atazanavir. Results from this two-drug combination study are summarized in Table 4 and suggest strong synergistic effects for indinavir and lopinavir, moderate synergy for nelfinavir, and additive to synergistic for atazanavir, amprenavir and saquinavir. Combining amprenavir with Compound 1 results in additivity at molar ratios of 1:6.7 and 1:16.7, with strong synergy at a 1:2.7 molar ratio. Combining saquinavir with Compound 1 yields additive responses at the 1:1.3 and 1:3.3 molar ratios, and strong synergy at 1:8.3. In general, protease combinations with Compound 1 are synergistic to additive. No cytotoxicity was observed at the highest concentrations used in any of these combination antiviral assays.

TABLE 4
Two-Drug Combination using Compound 1 and Protease Inhibitors

| | Combination Indices at % HIV Inhibition^{b} | | | |
|---|---|---|---|---|
| Molar Ratio | (Confidence Interval) | | | Overall |
| (EC₅₀ Ratio)^{a} | 50% | 75% | 90% | Result |

Indinavir

| | | | | | |
|---|---|---|---|---|---|
| 1:3.3 | (1:1) | 0.55 (0.46, 0.64) | 0.57 (0.45, 0.70) | 0.61 (0.39, 0.83) | Synergistic |
| 1:8.33 | (1:2.5) | 0.35 (0.24, 0.47) | 0.21 (0.17, 0.24) | 0.32 (0.20, 0.43) | |
| 1:1.3 | (2.5:1) | 0.31 (0.22, 0.40) | 0.41 (0.27, 0.55) | 0.54 (0.20, 0.87) | |

Lopinavir

| | | | | | |
|---|---|---|---|---|---|
| 1:6.7 | (1:1) | 0.23 (0.16, 0.42) | 0.22 (0.14, 0.30) | 0.26 (0.09, 0.44) | Synergistic |
| 1:16.7 | (1:2.5) | 0.13 (0.08, 0.18) | 0.06 (0.05, 0.08) | 0.03 (0.02, 0.05) | |
| 1:2.7 | (2.5:1) | 1.13 (0.73, 1.52) | 0.50 (0.26, 0.74) | 0.30 (0.07, 0.52) | |

Nelfinavir

| | | | | | |
|---|---|---|---|---|---|
| 1:3.3 | (1:1) | 0.66 (0.60, 0.73) | 0.78 (0.67, 0.88) | 0.91 (0.71, 1.10) | Moderate |
| 1:8.3 | (1:2.5) | 0.29 (0.31, 0.46) | 0.62 (0.45, 0.79) | 0.99 (0.57, 1.41) | Synergistic |
| 1:1.3 | (2.5:1) | 0.77 (0.69, 0.86) | 0.69 (0.62, 0.77) | 0.62 (0.49, 0.75) | |

Atazanavir

| | | | | | |
|---|---|---|---|---|---|
| 1:1 | (1:1) | 1.04 (0.60, 1.49) | 0.58 (0.24, 0.93) | 0.33 (0.02, 0.64) | Additive- |
| 1:2.5 | (1:2.5) | 0.23 (0.09, 0.38) | 0.35 (0.07, 0.63) | 0.52 (0.00, 1.24) | Synergistic |
| 1:0.4 | (2.5:1) | 0.88 (0.49, 1.27) | 0.67 (0.26, 1.09) | 0.52 (0.01, 1.04) | |

Amprenavir

| | | | | | |
|---|---|---|---|---|---|
| 1:6.7 | (1:1) | 0.89 (0.73, 1.06) | 0.93 (0.68, 1.17) | 0.96 (0.57, 1.36) | Additive- |
| 1:16.7 | (1:2.5) | 1.30 (0.84, 1.76) | 1.08 (0.55, 1.62) | 0.90 (0.20, 1.60) | Synergistic |
| 1:2.7 | (2.5:1) | 0.41 (0.34, 0.48) | 0.32 (0.25, 0.40) | 0.25 (0.16, 0.35) | |

Saquinavir

| | | | | | |
|---|---|---|---|---|---|
| 1:3.3 | (1:1) | 0.97 (0.88, 1.07) | 0.92 (0.76, 1.07) | 0.87 (0.65, 1.09) | Additive- |
| 1:8.3 | (1:2.5) | 0.22 (0.20, 0.24) | 0.26 (0.22, 0.29) | 0.29 (0.24, 0.35) | Synergistic |
| 1:1.3 | (2.5:1) | 1.07 (0.95, 1.20) | 0.92 (0.74, 1.10) | 0.80 (0.45, 1.14) | |

Ritonavir
^{a}Ratio of Compound 1 to comparator compound.
^{b}A lower bound of the asymptotic confidence interval greater than 1 indicates antagonisms, an upper bound of less than 1 indicates synergism, and a value of 1 being contained in the interval indicates additivity. The 95% confidence intervals are shown in parenthesis, and represent a measure of variability in the data.

Two-Drug Combination of Compound 1 with Entry Inhibitors. Enfuvirtide (T-20) is a recently approved HIV gp41 fusion inhibitor and the first approved Entry class inhibitor. The results presented in Table 5 indicate that the combination of the two entry inhibitors is synergistic. The only suggestion of additivity is observed at the 90% effective level for the molar ratio of 1:2.4. This result agrees with a similar study showing potent synergy for the combination of the gp120 antagonist PRO542 and enfuvirtide. No significant cytotoxicity was observed at the highest concentration of the combined drugs. The second HIV-1 gp41 antagonist, T-1249 also shows synergy when combined with compound 1. Along with the fusion inhibitors, two other classes of entry inhibitors were evaluated in combination with compound 1. AMD-3100 is a CXCR4 inhibitor, and it shows a strong synergy in combination with the attachment inhibitor. Sch-C and Sch-D are CCR5 inhibitors. They also exhibit moderate synergistic to synergistic response when combined with compound 1. None of these agents was cytotoxic at the concentrations studied.

TABLE 5
Anti-HIV Activity from a Two-Drug Combination
using Compound 1 and Entry Inhibitors.

| | Combination Indices at % HIV Inhibition^{b} | | | |
|---|---|---|---|---|
| Molar Ratio | (Confidence Interval) | | | Overall |
| (EC₅₀ Ratio)^{a} | 50% | 75% | 90% | Result |

Enfuvirtide

| | | | | | |
|---|---|---|---|---|---|
| 1:6 | (1:1) | 0.53 (0.46, 0.59) | 0.51 (0.42, 0.59) | 0.57 (0.42, 0.72) | Synergistic |
| 1:15 | (1:2.5) | 0.27 (0.21, 0.32) | 0.32 (0.22, 0.42) | 0.43 (0.22, 0.64) | |
| 1:2.4 | (2.5:1) | 0.41 (0.30, 0.51) | 0.57 (0.37, 0.78) | 0.90 (0.36, 1.44) | |

T-1249

| | | | | | |
|---|---|---|---|---|---|
| 1:0.67 | (1:1) | 0.25 (0.20, 0.30) | 0.14 (0.10, 0.19) | 0.08 (0.04, 0.12) | Synergistic |
| 1:1.67 | (1:2.5) | 0.32 (0.28 0.35) | 0.17 (0.14, 0.19) | 0.09 (0.06, 0.11) | |
| 1:0.27 | (2.5:1) | 0.19 (0.17, 0.22) | 0.14 (0.12, 0.16) | 0.10 (0.08, 0.13) | |

AMD-3100

| | | | | | |
|---|---|---|---|---|---|
| 1:6.67 | (1:1) | 0.40 (0.34, 0.46) | 0.27 (0.21, 0.33) | 0.18 (0.12, 0.24) | Synergistic |
| 1:16.67 | (1:2.5) | 0.25 (0.21, 0.29) | 0.14 (0.11, 0.17) | 0.08 (0.05, 0.10) | |
| 1:2.67 | (2.5:1) | 0.15 (0.11, 0.20) | 0.28 (0.18, 0.38) | 0.51 (0.20, 0.83) | |

Sch-D

| | | | | | |
|---|---|---|---|---|---|
| 1:0.17 | (1:1) | 0.49 (0.43, 0.55) | 0.41 (0.34, 0.49) | 0.36 (0.26, 0.46) | Synergistic |
| 1:0.42 | (1:2.5) | 0.71 (0.62, 0.81) | 0.55 (0.44, 0.65) | 0.44 (0.31, 0.57) | |
| 1:0.07 | (2.5:1) | 0.51 (0.45, 0.56) | 0.55 (0.47, 0.63) | 0.61 (0.46, 0.75) | |

Sch-C

| | | | | | |
|---|---|---|---|---|---|
| 1:1 | (1:1) | 0.63 (0.51, 0.74) | 0.70 (0.52, 0.89) | 0.80 (0.46, 1.13) | Moderate |
| 1:2.5 | (1:2.5) | 0.49 (0.41, 0.58) | 0.65 (0.49, 0.80) | 0.86 (0.52, 1.19) | Synergistic |
| 1:0.4 | (2.5:1) | 0.24 (0.19, 0.28) | 0.36 (0.27, 0.45) | 0.55 (0.31, 0.79) | |

UK-427,857
^{a}Ratio of Compound 1 to comparator compound.
^{b}A lower bound of the asymptotic confidence interval greater than 1 indicates antagonisms, an upper bound of less than 1 indicates synergism, and a value of 1 being contained in the interval indicates additivity. The 95% confidence intervals are shown in parenthesis, and represent a measure of variability in the data.

Two-Drug Combination of Compound 1 with an HIV integrase inhibitor. Compound 1 was tested with Compound 2 and the results presented in Table 6 indicate that the combination of the two inhibitors is additive-synergistic. No significant cytotoxicity was observed at the highest concentration of the combined drugs.

TABLE 6
Anti-HIV Activity from a Two-Drug Combination
using Compound 1 and Compound 2

| | Combination Indices at % HIV Inhibition^{b} | | | |
|---|---|---|---|---|
| Molar Ratio | (Confidence Interval) | | | Overall |
| (EC₅₀ Ratio)^{a} | 50% | 75% | 90% | Result |

Compound 2

| | | | | |
|---|---|---|---|---|
| 1:33.3 (1:1) | 0.92 (0.77, 1.06) | 0.90 (0.70, 1.10) | 0.89 (0.58, 1.21) | Additive- |
| 1:83.3 (1:2.5) | 0.71 (0.60, 0.82) | 0.66 (0.51, 0.80) | 0.61 (0.40, 0.82) | Synergistic |
| 1:13.3 (2.5:1) | 0.41 (0.36, 0.47) | 0.41 (0.33, 0.48) | 0.40 (0.28, 0.52) | |

^{a}Ratio of Compound 1 to comparator compound.
^{b}A lower bound of the asymptotic confidence interval greater than 1 indicates antagonisms, an upper bound of less than 1 indicates synergism, and a value of 1 being contained in the interval indicates additivity. The 95% confidence intervals are shown in parenthesis, and represent a measure of variability in the data.

### Pharmaceutical Composition and Methods of Use

Compound 1 inhibits HIV attachment, an essential step in HIV replication, and can be useful for the treatment of HIV infection and the consequent pathological conditions such as AIDS or ARC. As shown above, Compound 1 is active in conjunction with a wide variety of other agents and may be particularly beneficial in HAART and other new combination compositions and therapies.

Compound 1 will generally be given as a pharmaceutical composition, and the active ingredient of the composition may be comprised of Compound 1 alone or Compound 1 and at least one other agent used for treating AIDS or HIV infection. The compositions will generally be made with a pharmaceutically accepted carrier or vehicle, and may contain conventional exipients. The compositions are made using common formulation techniques. The invention encompasses all conventional forms. Solid and liquid compositions are preferred. Some solid forms include powders, tablets, capsules, and lozenges. Tablets include chewable, buffered, and extended release. Capsules include enteric coated and extended release capsules. Powders are for both oral use and reconstitution into solution. Powders include lyophilized and flash-melt powders. In a solid composition, Compound 1 and any antiretroviral agent are present in dosage unit ranges. Generally, Compound 1 will be in a unit dosage range of 1-1000 mg/unit. Some examples of dosages are 1 mg, 10, mg, 100, mg, 250 mg, 500 mg, and 1000 mg. Generally, other antiretroviral agents will be present in a unit range similar to agents of that class used clinically. Typically, this is 0.25-1000 mg/unit.

Liquids include aqueous solutions, syrups, elixers, emusions, and suspensions. In a liquid composition, Compound 1 and any antiretroviral agent are present in dosage unit ranges. Generally, Compound 1 will be in a unit dosage range of 1-100 mg/mL. Some examples of dosages are 1 mg/mL, 10 mg/mL, 25, mg/mL, 50 mg/mL, and 100 mg/mL. Generally, other antiretroviral agents will be present in a unit range similar to agents of that class used clinically. Typically, this is 1-100 mg/mL.

The invention encompasses all conventional modes of administration; oral and parenteral (injected intramuscular, intravenous, subcutanaeous) methods are preferred. Generally, the dosing regimen will be similar to other antiretroviral agents used clinically. Typically, the daily dose will be 1-100 mg/kg body weight daily for Compound 1. Generally, more compound is required orally and less parenterally. The specific dosing regime, however, will be determined by a physician using sound medical judgement.

The invention also encompasses methods where Compound 1 is given in combination therapy. That is, Compound 1 can be used in conjunction with, but separately from, other agents useful in treating AIDS and HIV infection. Some of these agents include HIV attachment inhibitors, CCR5 inhibitors, CXCR4 inhibitors, HIV cell fusion inhibitors, HIV integrase inhibitors, HIV nucleoside reverse transcriptase inhibitors, HIV non-nucleoside reverse transcriptase inhibitors, HIV protease inhibitors, budding and maturation inhibitors, immunomodulators, and anti-infectives. In these combination methods, Compound 1 will generally be given in a daily dose of 1-100 mg/kg body weight daily in conjunction with other agents. The other agents generally will be given in the amounts used therapeutically. The specific dosing regime, however, will be determined by a physician using sound medical judgement.

Table 7 lists some agents useful in treating AIDS and HIV infection, which are suitable for this invention. The invention, however, is not limited to these agents.

TABLE 7

| | | |
|---|---|---|
| DRUG NAME | MANUFACTURER | INDICATION |

ANTIVIRALS

| | | |
|---|---|---|
| 097 | Hoechst/Bayer | HIV infection, AIDS, |
| (non-nucleoside reverse | | ARC |
| transcriptase inhibitor) | | |
| Amprenavir | Glaxo Wellcome | HIV infection, AIDS, |
| 141 W94 | | ARC |
| GW 141 | | |
| (protease inhibitor) | | |
| Abacavir (1592U89) | Glaxo Wellcome | HIV infection, AIDS, |
| GW 1592 | | ARC |
| (RT inhibitor) | | |
| Acemannan | Carrington Labs | ARC |
| | (Irving, TX) | |
| Acyclovir | Burroughs Wellcome | HIV infection, AIDS, |
| | | ARC, in combination |
| | | with AZT |
| AD-439 | Tanox Biosystems | HIV infection, AIDS, |
| | | ARC |
| AD-519 | Tanox Biosystems | HIV infection, AIDS, |
| | | ARC |
| Adefovir dipivoxil | Gilead Sciences | HIV infection, ARC, |
| AL-721 | Ethigen | PGL HIV positive, AIDS |
| | (Los Angeles, CA) | |
| Alpha Interferon | Glaxo Wellcome | Kaposi's sarcoma |
| HIV in combination | | |
| w/Retrovir | | |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| Neutralizes pH | Concepts | |
| Labile alpha aberrant | (Rockville, MD) | |
| Interferon | | |
| AR177 | Aronex Pharm | HIV infection, AIDS, |
| | | ARC |
| Beta-fluoro-ddA | Nat'l Cancer Institute | AIDS-associated |
| | | diseases |
| BMS-232623 | Bristol-Myers Squibb/ | HIV infection, AIDS, |
| (CGP-73547) | Novartis | ARC |
| (protease inhibitor) | | |
| BMS-234475 | Bristol-Myers Squibb/ | HIV infection, AIDS, |
| (CGP-61755) | Novartis | ARC |
| (protease inhibitor) | | |
| CI-1012 | Warner-Lambert | HIV-1 infection |
| Cidofovir | Gilead Science | CMV retinitis, herpes, |
| | | papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening |
| Ganciclovir | | CMV peripheral, CMV |
| | | retinitis |
| Delaviridine | Pharmacia-Upjohn | HIV infection, AIDS, |
| (RT inhibitor) | | ARC |
| Dextran Sulfate | Ueno Fine Chem. | AIDS, ARC, HIV |
| | Ind. Ltd. (Osaka, | positive asymptomatic |
| | Japan) | |
| ddC | Hoffman-La Roche | HIV infection, AIDS, |
| Dideoxycytidine | | ARC |
| ddI | Bristol-Myers Squibb | HIV infection, AIDS, |
| Dideoxyinosine | | ARC; combinationwith |
| | | AZT/d4T |
| DMP-450 | AVID (Camden, NJ) | HIV infection, AIDS, |
| (protease inhibitor) | | ARC |
| Efavirenz | DuPont Merck | HIV infection, AIDS, |
| (DMP 266) | | ARC |
| (−)6-Chloro-4-(S)- | | |
| cyclopropylethynyl- | | |
| 4(S)-trifluoro- | | |
| methyl-1,4-dihydro- | | |
| 2H-3,1-benzoxazin- | | |
| 2-one, STOCRINE | | |
| (non-nucleoside RT | | |
| inhibitor) | | |
| EL10 | Elan Corp, PLC | HIV infection |
| | (Gainesville, GA) | |
| Famciclovir | Smith Kline | herpes zoster, herpes |
| | | simplex |
| FTC (reverse | Emory University | HIV infection, AIDS, |
| transcriptase | | ARC |
| inhibitor) | | |
| GS 840 | Gilead | HIV infection, AIDS, |
| (reverse transcriptase | | ARC |
| inhibitor) | | |
| HBY097 | Hoechst Marion | HIV infection, AIDS, |
| (non-nucleoside reverse | Roussel | ARC |
| transcriptase inhibitor) | | |
| Hypericin | VIMRx Pharm. | HIV infection, AIDS, |
| | | ARC |
| Recombinant Human | Triton Biosciences | AIDS, Kaposi's sarcoma, |
| Interferon Beta | (Almeda, CA) | ARC |
| Interferon alfa-n3 | Interferon Sciences | ARC, AIDS |
| Indinavir | Merck | HIV infection, AIDS, |
| | | ARC, asymptomatic HIV |
| | | positive, also in |
| | | combination with |
| | | AZT/ddI/ddC |
| ISIS 2922 | ISIS Pharmaceuticals | CMV retinitis |
| KNI-272 | Nat'l Cancer Institute | HIV-associated |
| | | diseases |
| Lamivudine, 3TC | Glaxo Wellcome | HIV infection, AIDS, |
| (reverse transcriptase | | ARC, also with AZT |
| inhibitor) | | |
| Lobucavir | Bristol-Myers Squibb | CMV infection |
| Nelfinavir | Agouron | HIV infection, AIDS, |
| (protease inhibitor) | Pharmaceuticals | ARC |
| Nevirapine | Boeheringer | HIV infection, AIDS, |
| (RT inhibitor) | Ingleheim | ARC |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc. | infection, other CMV |
| | | infections |
| PNU-140690 | Pharmacia Upjohn | HIV infection, AIDS, |
| (protease inhibitor) | | ARC |
| Probucol | Vyrex | HIV infection, AIDS |
| RBC-CD4 | Sheffield Med. | HIV infection, AIDS, |
| | Tech (Houston, TX) | ARC |
| Ritonavir | Abbott | HIV infection, AIDS, |
| (protease inhibitor) | | ARC |
| Saquinavir | Hoffmann- | HIV infection, AIDS, |
| (protease inhibitor) | LaRoche | ARC |
| Stavudine; d4T | Bristol-Myers Squibb | HIV infection, AIDS, |
| Didehydrodeoxy- | | ARC |
| thymidine | | |
| Valaciclovir | Glaxo Wellcome | Genital HSV & |
| | | CMV infections |
| Virazole | Viratek/ICN | asymptomatic HIV- |
| Ribavirin | (Costa Mesa, CA) | positive, LAS, ARC |
| VX-478 | Vertex | HIV infection, AIDS, |
| | | ARC |
| Zalcitabine | Hoffmann-LaRoche | HIV infection, AIDS, |
| | | ARC, with AZT |
| Zidovudine; AZT | Glaxo Wellcome | HIV infection, AIDS, |
| | | ARC, Kaposi's sarcoma, |
| | | in combination with |
| | | other therapies |
| Tenofovir disoproxil, | Gilead | HIV infection, AIDS |
| fumarate salt | | |
| (Viread ®) | | |
| (reverse transcriptase | | |
| inhibitor) | | |
| Combivir ® | GSK | HIV infection, AIDS |
| (reverse transcriptase | | |
| inhibitor) | | |
| abacavir succinate | GSK | HIV infection, AIDS |
| (or Ziagen ®) | | |
| (reverse transcriptase | | |
| inhibitor) | | |
| Reyataz ® | Bristol-Myers Squibb | HIV infection, AIDS |
| (atazanavir) | | |
| Fuzeon | Roche/Trimeris | HIV infection, AIDS, |
| (Enfuvirtide, T-20) | | viral fusion inhibitor |
| Trizivir ® | | HIV infection, AIDS |
| Kaletra ® | Abbott | HIV infection, AIDS, |
| | | ARC |

IMMUNOMODULATORS

| | | |
|---|---|---|
| AS-101 | Wyeth-Ayerst | AIDS |
| Bropirimine | Pharmacia Upjohn | Advanced AIDS |
| Acemannan | Carrington Labs, Inc. | AIDS, ARC |
| | (Irving, TX) | |
| CL246,738 | American Cyanamid | AIDS, Kaposi's sarcoma |
| | Lederle Labs | |
| EL10 | Elan Corp, PLC | HIV infection |
| | (Gainesville, GA) | |
| FP-21399 | Fuki ImmunoPharm | Blocks HIV fusion with |
| | | CD4+ cells |
| Gamma Interferon | Genentech | ARC, in combination |
| | | w/TNF (tumor necrosis |
| | | factor) |
| Granulocyte | Genetics Institute | AIDS |
| Macrophage Colony | Sandoz | |
| Stimulating Factor | | |
| Granulocyte | Hoechst-Roussel | AIDS |
| Macrophage Colony | Immunex | |
| Stimulating Factor | | |
| Granulocyte | Schering-Plough | AIDS, combination |
| Macrophage Colony | | w/AZT |
| Stimulating Factor | | |
| HIV Core Particle | Rorer | Seropositive HIV |
| Immunostimulant | | |
| IL-2 | Cetus | AIDS, in combination |
| Interleukin-2 | | w/AZT |
| IL-2 | Hoffman-LaRoche | AIDS, ARC, HIV, in |
| Interleukin-2 | Immunex | combination w/AZT |
| IL-2 | Chiron | AIDS, increase in CD4 |
| Interleukin-2 | | cell counts |
| (aldeslukin) | | |
| Immune Globulin | Cutter Biological | Pediatric AIDS, in |
| Intravenous | (Berkeley, CA) | combination w/AZT |
| (human) | | |
| IMREG-1 | Imreg | AIDS, Kaposi's sarcoma, |
| | (New Orleans, LA) | ARC, PGL |
| IMREG-2 | Imreg | AIDS, Kaposi's sarcoma, |
| | (New Orleans, LA) | ARC, PGL |
| Imuthiol Diethyl | Merieux Institute | AIDS, ARC |
| Dithio Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma AIDS, |
| Muramyl-Tripeptide | Amgen | in combination w/AZT |
| Granulocyte | | |
| Colony Stimulating | | |
| Factor | | |
| Remune | Immune Response | Immunotherapeutic |
| | Corp. | |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma, AIDS, |
| Alfa 2a | in combination w/AZT | ARC |
| SK&F106528 | Smith Kline | HIV infection |
| Soluble T4 | | |
| Thymopentin | Immunobiology | HIV infection |
| | Research Institute | |
| | (Annandale, NJ) | |
| Tumor Necrosis | Genentech | ARC, in combination |
| Factor; TNF | | w/gamma Interferon |

ANTI-INFECTIVES

| | | |
|---|---|---|
| Clindamycin with | Pharmacia Upjohn | PCP |
| Primaquine | | |
| Fluconazole | Pfizer | Cryptococcal |
| | | meningitis, |
| | | candidiasis |
| Pastille | Squibb Corp. | Prevention of oral |
| Nystatin Pastille | | candidiasis |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | Antibacterial |
| Trimethoprim/sulfa | | Antibacterial |
| Piritrexim | Burroughs Wellcome | PCP treatment |
| Pentamidine | Fisons Corporation | PCP prophylaxis |
| Isethionate for | | |
| Inhalation | | |
| Spiramycin | Rhone-Poulenc | Cryptosporidial |
| | diarrhea | |
| Intraconazole- | Janssen-Pharm. | Histoplasmosis; |
| R51211 | | cryptococcal |
| | | meningitis |
| Trimetrexate | Warner-Lambert | PCP |
| Daunorubicin | NeXstar, Sequus | Kaposi's sarcoma |
| Recombinant Human | Ortho Pharm. Corp. | Severe anemia assoc. |
| Erythropoietin | | with AZT therapy |
| Recombinant Human | Serono | AIDS-related wasting, |
| Growth Hormone | | cachexia |
| Megestrol Acetate | Bristol-Myers Squibb | Treatment of anorexia |
| | | assoc. W/AIDS |
| Testosterone | Alza, Smith Kline | AIDS-related wasting |
| Total Enteral | Norwich Eaton | Diarrhea and |
| Nutrition | Pharmaceuticals | malabsorption related |
| | | to AIDS |

1. A method for treating HIV infection in a human patient comprising administering a therapeutically effective amount of 1-benzoyl-4-[2-(4,7-dimethoxy-1H-pyrrolo[2,3-c]pyridin-3-yl)-1,2-dioxoethyl]-piperazine or a pharmaceutically acceptable salt or solvate thereof with a therapeutically effective amount of at least one other agent used for treatment of AIDS or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

2. The method of claim 1 wherein the agent is a nucleoside HIV reverse transcriptase inhibitor.

3. The method of claim 2 wherein the nucleoside HIV reverse transcriptase inhibitor is selected from the group consisting of abacavir, didanosine, emtricitabine, lamivudine, stavudine, tenofovir, zalcitabine, and zidovudine, or a pharmaceutically acceptable salt or solvate thereof.

4. The method of claim 1 wherein the agent is a non-nucleoside HIV reverse transcriptase inhibitor.

5. The method of claim 4 wherein the non-nucleoside HIV reverse transcriptase inhibitor is selected from the group consisting of delavirdine, efavirenz, and nevirapine, or a pharmaceutically acceptable salt or solvate thereof.

6. The method of claim 1 wherein the agent is an HIV protease inhibitor.

7. The method of claim 6 wherein the HIV protease inhibitor is selected from the group consisting of amprenavir, atazanavir, indinavir, lopinavir, nelfinavir, ritonavir, saquinavir and fosamprenavir, or a pharmaceutically acceptable salt or solvate thereof.

8. The method of claim 1 wherein the agent is an HIV fusion inhibitor.

9. The method of claim 8 wherein the HIV fusion inhibitor is enfuvirtide or T-1249, or a pharmaceutically acceptable salt or solvate thereof.

10. The method of claim 1 wherein the agent is an HIV attachment inhibitor.

11. The method of claim 1 wherein the agent is a CCR5 inhibitor.

12. The method of claim 11 wherein the CCR5 inhibitor is selected from the group consisting of Sch-C, Sch-D, TAK-220, PRO-140, and UK-427,857, or a pharmaceutically acceptable salt or solvate thereof.

13. The method of claim 1 wherein the agent is a CXCR4 inhibitor.

14. The method of claim 13 wherein the CXCR4 inhibitor is AMD-3100, or a pharmaceutically acceptable salt or solvate thereof.

15. The method of claim 1 wherein the agent is an HIV budding or maturation inhibitor.

16. The method of claim 15 wherein the budding or maturation inhibitor is PA-457, or a pharmaceutically acceptable salt or solvate thereof.

17. The method of claim 1 wherein the agent is an HIV integrase inhibitor.

18. The method of claim 17 wherein the HIV integrase inhibitor is 3-[(4-fluorobenzyl)methoxycarbamoyl]-2-hydroxyacrylic acid or 2-(2,2)-dimethyl-5-oxo-[1,3]-dioxolan-4-ylidene)-N-(4-fluorobenzyl)-N-methoxyacetamide, or a pharmaceutically acceptable salt or solvate thereof.

19. A pharmaceutical composition comprising a therapeutically effective amount of 1-benzoyl-4-[2-(4,7-dimethoxy-1H-pyrrolo[2,3-c]pyridin-3-yl)-1,2-dioxoethyl]-piperazine, or a pharmaceutically acceptable salt or solvate thereof, with at least one other agent used for treatment of AIDS or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier.

20. The composition of claim 19 wherein the agent is a nucleoside HIV reverse transcriptase inhibitor.

21. The composition of claim 20 wherein the nucleoside HIV transcriptase inhibitor is selected from the group consisting of abacavir, didanosine, emtricitabine, lamivudine, stavudine, tenofovir, zalcitabine, and zidovudine, or a pharmaceutically acceptable salt or solvate thereof.

22. The composition of claim 19 wherein the agent is a non-nucleoside HIV reverse transcriptase inhibitor.

23. The composition of claim 22 wherein the non-nucleoside HIV reverse transcriptase inhibitor is selected from the group consisting of delavirdine, efavirenz, and nevirapine, or a pharmaceutically acceptable salt or solvate thereof.

24. The composition of claim 19 wherein the agent is an HIV protease inhibitor.

25. The composition of claim 24 wherein the HIV protease inhibitor is selected from the group consisting of amprenavir, atazanavir, indinavir, lopinavir, nelfinavir, ritonavir, saquinavir and fosamprenavir, or a pharmaceutically acceptable salt or solvate thereof.

26. The composition of claim 19 wherein the agent is an HIV fusion inhibitor.

27. The composition of claim 26 wherein the HIV fusion inhibitor is enfuvirtide or T-1249, or a pharmaceutically acceptable salt or solvate thereof.

28. The composition of claim 19 wherein the agent is an HIV attachment inhibitor.

29. The composition of claim 19 wherein the agent is a CCR5 inhibitor.

30. The composition of claim 29 wherein the CCR5 inhibitor is selected from the group consisting of Sch-C, Sch-D, TAK-220, PRO-140, and UK-427,857, or a pharmaceutically acceptable salt or solvate thereof.

31. The composition of claim 19 wherein the agent is a CXCR4 inhibitor.

32. The composition of claim 31 wherein the CXCR4 inhibitor is AMD-3100, or a pharmaceutically acceptable salt or solvate thereof.

33. The composition of claim 19 wherein the agent is an HIV budding or maturation inhibitor.

34. The composition of claim 33 wherein the budding or maturation inhibitor is PA-457, or a pharmaceutically acceptable salt or solvate thereof.

35. The composition of claim 19 wherein the agent is an HIV integrase inhibitor.

36. The composition of claim 35 wherein the HIV integrase inhibitor is 3-[(4-fluorobenzyl)methoxycarbamoyl]-2-hydroxyacrylic acid or 2-(2,2)-dimethyl-5-oxo-[1,3]-dioxolan-4-ylidene)-N-(4-fluorobenzyl)-N-methoxyacetamide, or a pharmaceutically acceptable salt or solvate thereof.
